(19) United States
(12) Patent Application Publication
(10) Pub. No.: US 2026/0263242 A1
(43) Pub. Date: Sep. 10, 2026

(54) IMPLANTABLE MEDICAL DEVICE HAVING AN ELECTRONIC PUMP DEVICE WITH A PASSIVE VALVE LAYER ON A BASE PLATE

(71) Applicant: Boston Scientific Scimed, Inc., Maple Grove, MN (US)

(72) Inventors: Richard Percy, Leamlara (IE); Thomas Sinnott, Enniscorthy (IE); Brian P. Watschke, Minneapolis, MN (US)

(21) Appl. No.: 19/549,690

(22) Filed: Feb. 25, 2026

Related U.S. Application Data
(60) Provisional application No. 63/766,712, filed on Mar. 4, 2025.

(51) Int. Cl. A61F 2/48; A61F 2/00; A61F 2/26

(52) U.S. Cl. CPC A61F 2/484; A61F 2/004; A61F 2/26; A61F 2/482; A61F 2240/001; A61F 2250/0013

(57) ABSTRACT

According to an aspect, an implantable medical device includes an inflatable member, a fluid reservoir, and an electronic pump device configured to transfer fluid between the inflatable member and the fluid reservoir. The electronic pump device includes an actuator, a base plate having a first surface and a second surface, a first passive valve layer contacting the first surface of the base plate, and a second passive valve layer contacting the second surface of the base plate.

### CROSS-REFERENCE TO RELATED APPLICATION

This application claims priority to U.S. Provisional Patent Application No. 63/766,712, filed on Mar. 4, 2025, entitled “IMPLANTABLE MEDICAL DEVICE HAVING AN ELECTRONIC PUMP DEVICE WITH A PASSIVE VALVE LAYER ON A BASE PLATE”, the disclosure of which is incorporated by reference herein in its entirety.

### TECHNICAL FIELD

This disclosure relates generally to an implantable medical device having an electronic pump device with a passive valve layer on a base plate for improving the flow of fluid.

### BACKGROUND

Some inflatable medical devices have a pump device, which, when operated by a user, causes a transfer of fluid between a fluid reservoir and an inflatable member. The pump device may have fluidic components (e.g., pump(s) and/or valves), where a fluidic component may have multiple components. A location of a valve layer in the pump device may affect the performance of transferring fluid.

### SUMMARY

This disclosure relates to an inflatable medical device with an electronic pump device, wherein the electronic pump device includes an electronically controlled pump. The pump may include an actuator, a base plate, and passive valve layers defining an inlet valve and an outlet valve. The actuator may include an activation element and an actuator diaphragm coupled to the activation element. The activation element may receive an electrical signal, which causes the activation element to deform, thereby deforming the actuator diaphragm. The base plate includes a first surface and a second surface disposed opposite to the first surface. The first passive valve layer may be positioned on and contact the first surface of the base plate. The second passive valve layer may be positioned on and contact the second surface of the base plate. The location of the second passive valve layer (e.g., below the second surface of the base plate) may increase the stroke of the pump, which allows the pump to draw in and expel a larger volume of fluid with each cycle, thereby increasing the performance of the electronic pump device.

In some aspects, the techniques described herein relate to an implantable medical device including: an inflatable member; a fluid reservoir; and an electronic pump device configured to transfer fluid between the inflatable member and the fluid reservoir, the electronic pump device including: an actuator; a base plate having a first surface and a second surface, the first surface of the base plate facing the actuator; a passive valve layer contacting the second surface of the base plate.

In some aspects, the techniques described herein relate to an electronic pump device for an inflatable medical device, the electronic pump device including: an actuator; a base plate having a first surface and a second surface, the first surface of the base plate facing the actuator; a passive valve layer contacting the second surface of the base plate.

In some aspects, the techniques described herein relate to a method including: forming an electronically controlled pump of an inflatable medical device, including: disposing a first passive valve layer on a first surface of a base plate; and disposing a second passive valve layer on a second surface of the base plate, the electronically controlled pump including an actuator configured to receive an electrical signal; and attaching the electronically controlled pump to a fluidic manifold of the inflatable medical device.

In some aspects, the techniques described herein relate to an implantable medical device including: an inflatable member; a fluid reservoir; and an electronic pump device configured to transfer fluid between the inflatable member and the fluid reservoir, the electronic pump device including: an actuator; a base plate having a first surface and a second surface, the first surface of the base plate facing the actuator; a passive valve layer contacting the second surface of the base plate.

In some aspects, the techniques described herein relate to an electronic pump device for an inflatable medical device, the electronic pump device including: an actuator having an activation element and an actuator diaphragm coupled to the activation element; a base plate having a first surface and a second surface; a first passive valve layer contacting the first surface of the base plate, the first passive valve layer being located between the base plate and the actuator diaphragm; and a second passive valve layer contacting the second surface of the base plate.

In some aspects, the techniques described herein relate to a method including: forming an electronically controlled pump of an inflatable medical device, including: disposing a first passive valve layer on a first surface of a base plate; and disposing a second passive valve layer on a second surface of the base plate, the electronically controlled pump including an actuator configured to receive an electrical signal; and attaching the electronically controlled pump to a fluidic manifold of the inflatable medical device.

### BRIEF DESCRIPTION OF THE DRAWINGS

FIG. 1 illustrates an implantable medical device with an electronically controlled pump that includes a passive valve layer contacting a surface of a base plate that is opposite to an actuator according to an aspect.

FIG. 2A illustrates an example of a pump of an inflatable medical device according to an aspect.

FIG. 2B illustrates an example of a pump of an inflatable medical device according to another aspect.

FIG. 2C illustrates an example of a double valve according to an aspect.

FIG. 3 illustrates an example of a pump of an inflatable medical device according to another aspect.

FIG. 4 illustrates an example of a pump of an inflatable medical device according to another aspect.

FIG. 5 illustrates an example of a pump of an inflatable medical device according to another aspect.

FIG. 6 illustrates an example of a pump of an inflatable medical device according to another aspect.

FIG. 7A an example of a fluidic manifold configured to attach a pump of an inflatable medical device according to an aspect.

FIG. 7B illustrates a circuit substrate coupled to the fluidic manifold according to an aspect.

FIG. 7C illustrates a perspective of the fluidic manifold according to an aspect.

FIG. 7D illustrates a perspective of the fluidic manifold according to another aspect.

FIG. 7E illustrates a perspective of a circuit substrate coupled to a fluidic manifold according to an aspect.

FIG. 8 illustrates an inflatable medical device according to an aspect.

FIG. 9 illustrates an inflatable medical device according to another aspect.

FIG. 10 illustrates an exploded view of a housing of an electronic pump device according to an aspect.

FIG. 11 illustrates a perspective of an inflatable penile prosthesis according to an aspect.

FIG. 12 illustrates an example of an artificial urinary sphincter device according to an aspect.

FIG. 13 illustrates a flowchart depicting example operations of forming an electronically controlled pump according to an aspect.

### DETAILED DESCRIPTION

This disclosure relates to an implantable medical device with an electronic pump device configured to automatically transfer fluid between a fluid reservoir and an inflatable member. In some examples, the implantable medical device includes a urology device. In some examples, the implantable medical device includes a penile prosthesis with one or more inflatable cylinders. In some examples, the implantable medical device includes a urinary control device with an inflatable cuff. However, the implantable medical device may include other types of medical devices such as a stimulation device or a neuromodulation device. The electronic pump device includes a housing with a fluidic manifold having a fluidic component to facilitate the transfer of fluid between the fluid reservoir and the inflatable member. In some examples, the fluidic component includes a pump.

The pump includes an actuator (e.g., an activation element and an actuator diaphragm), a base plate, and passive valve layers defining an inlet valve and an outlet valve. The base plate includes a first surface and a second surface. The first passive valve layer may be positioned on and contact the first surface of the base plate. The second passive valve layer may be positioned on and contact the second surface of the base plate. The location of the second passive valve layer (e.g., below the second surface of the base plate) may increase the stroke of the pump, which allows the pump to draw in and expel a larger volume of fluid with each cycle, thereby increasing the performance of the electronic pump device. For example, at least a portion of the pump may be disposed within a recess of a fluidic manifold, and a portion of the actuator may be coupled (e.g., welded) to a surface portion of a fluidic manifold. By positioning one or more of the passive valve layers below the base plate, the motion of the actuator diaphragm may be larger, thereby increasing the stroke length.

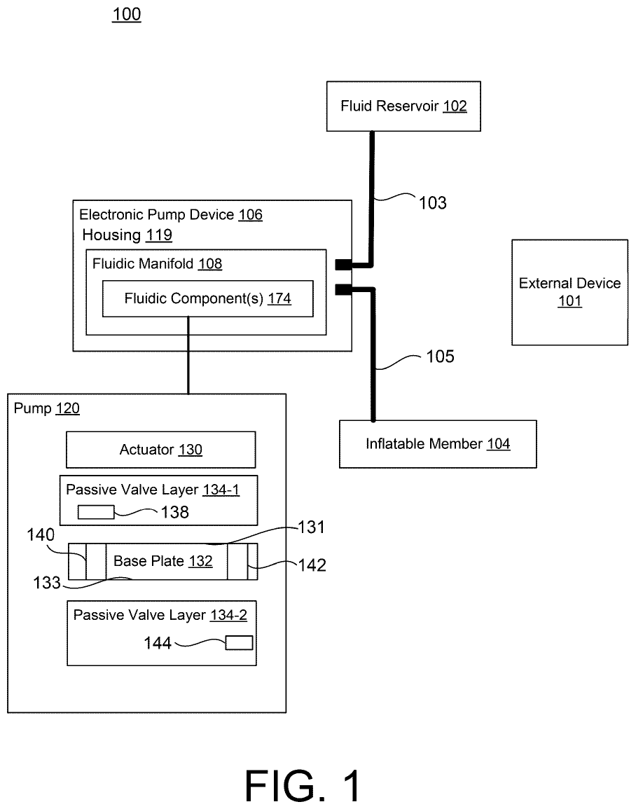

FIG. 1 illustrates an implantable medical device 100 according to an aspect. In some examples, the implantable medical device 100 includes a urology device. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100 such as respiratory devices, cardiovascular devices, gastrointestinal devices, ophthalmic devices, drug delivery devices, and/or diagnostic devices. In some examples, the implantable medical device 100 may be a stimulation device. In some examples, the implantable medical device 100 may be a neuromodulation device.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump device 106 may be implemented in the patient's body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

The electronic pump device 106 includes a housing 119. The housing 119 includes a fluidic manifold 108 that attaches fluid transfer and pressure regulating components. In some examples, the fluidic manifold 108 includes a conductive-based material (e.g., a metal-based material). In some examples, the fluidic manifold 108 is a titanium frame. The fluidic manifold 108 may attach a circuit substrate with electronic components. The fluidic manifold 108 may attach one or more fluidic components 174. A fluidic component 174 may be a pump 120. A fluidic component 174 may be a valve. In some examples, a pump 120 includes an electronically controlled pump. In some examples, the pump 120 includes a piezoelectric diaphragm pump (e.g., an example of an electronically controlled pump). In some examples, the fluidic manifold 108 includes a recess, and at least a portion of the pump 120 is located in the recess.

The pump 120 may include an actuator 130. The actuator 130 may receive an electrical signal, in response to the electrical signal, the actuator 130 may deform (e.g., bend, change its shape, etc.). The actuator 130 may be one or more multiple components that are coupled together. In some examples, the actuator 130 includes one or more disc-shaped elements. In some examples, the actuator 130 includes an activation element (e.g., a piezo element with one or more electrodes, a piezo disc actuator, etc.) and an actuator diaphragm coupled to the activation element. The actuator diaphragm may be a flexible membrane. In some examples, the actuator diaphragm may be a metal-based flexible membrane. In some examples, the actuator 130 includes a piezo disc actuator, an isolation layer, an adhesive material, and an actuator diaphragm. An electrical signal applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump 120.

The pump 120 may include a base plate 132. The base plate 132 may be a disc-shaped metal element. The base plate 132 may include a first hole 140 (e.g., a first through-hole) and a second hole 142 (e.g., a second through-hole). The base plate 132 includes a first surface 131 and a second surface 133 disposed opposed to the first surface. The distance between the first surface 131 and the second surface 133 may define a thickness of the base plate 132.

The pump 120 includes a passive valve layer 134-1 and a passive valve layer 134-2. In some examples, the passive valve layer 134-1 is a circular disc member (e.g., a thin metal-based disc). The passive valve layer 134-1 includes a fluid control element 138 at a location on the passive valve layer 134-1. The fluid control element 138 may be a feature on the passive valve layer 134-1 that causes fluid to flow through the passive valve layer 134-1 in one direction (but not the other direction). In some examples, the fluid control element 138 is a check valve. In some examples, the fluid control element 138 includes an input check valve. In some examples, the fluid control element 138 includes an output check valve. The fluid control element 138 may include one or more slots. In some examples, the fluid control element 138 includes a helical slot. In some examples, the fluid control element 138 is aligned with the first hole 140.

In some examples, the passive valve layer 134-2 is a circular disc member. The passive valve layer 134-2 includes a fluid control element 144 at a location on the passive valve layer 134-2. The fluid control element 144 may be a feature on the passive valve layer 134-2 that causes fluid to flow through the passive valve layer 134-2 in one direction (but not in the other direction). In some examples, the fluid control element 144 is a check valve. In some examples, the fluid control element 144 includes an input check valve. In some examples, the fluid control element 144 includes an output check valve. The fluid control element 144 may include one or more slots. In some examples, the fluid control element 144 includes a helical slot. In some examples, the fluid control element 144 is aligned with the second hole 142.

The passive valve layer 134-1 may contact (e.g., directly contact) the first surface 131 (or a portion thereof) of the base plate 132. In some examples, instead of contacting the first surface 131 of the base plate 132, the passive valve layer 134-1 is disposed below (e.g., contacts) the surface (e.g., the bottom surface) of the passive valve layer 134-2. The passive valve layer 134-2 may contact (e.g., directly contact) the second surface 133 (or a portion thereof) of the base plate 132. The location of the passive valve layer 134-2 (e.g., below the second surface 133) may increase the stroke of the pump 120. For example, a longer stroke (e.g., distance that the actuator diaphragm can travel) may allow the pump 120 to draw in and expel a larger volume of fluid with each cycle. In some examples, the pump 120 includes multiple passive valve layers 134-1 located between the actuator 130 and the first surface 131 of the base plate 132. In some examples, the pump 120 includes multiple passive valve layers 134-2 disposed below the second surface 133 of the base plate 132.

In some examples, the fluidic manifold 108 defines a recess (e.g., a circular opening in the frame of the fluidic manifold 108) configured to receive at least a portion of the pump 120. The layers of the pump 120 (e.g., the actuator 130, the passive valve layer 134-1, the base plate 132, and the passive valve layer 134-2) may form a stack, and one end of the stack (e.g., the passive valve layer 134-2) may contact a surface within the recess (e.g., a recessed surface). At least a portion of the actuator 130 may be coupled (e.g., welded) to a surface (e.g., outside the recess) of the fluidic manifold 108, thereby forming a pump chamber.

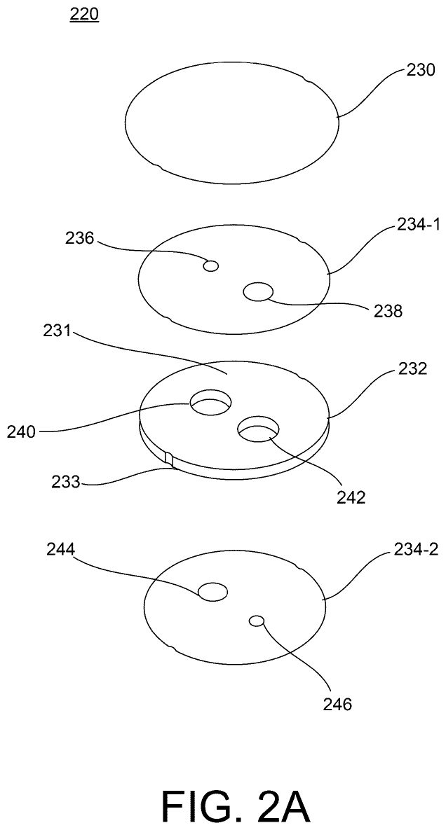

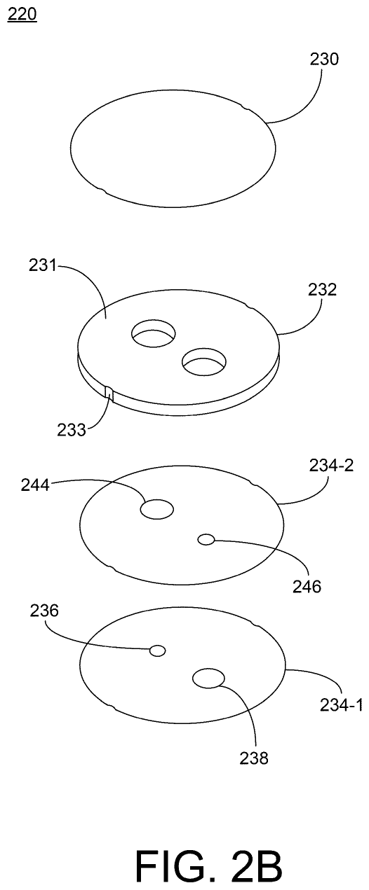

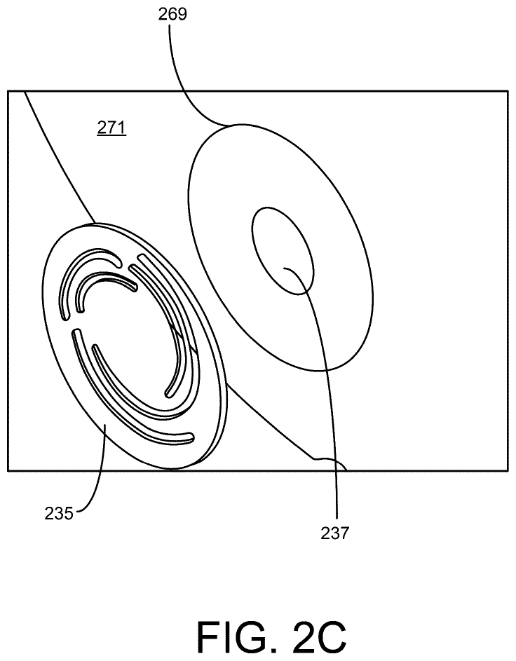

FIG. 2A illustrates a pump 220 according to an aspect. The pump 220 may be an example of the pump 120 of FIG. 1 and may include any of the details discussed herein. The pump 220 may include an actuator 230, a passive valve layer 134-1, a base plate 232, and a passive valve layer 134-2. The actuator 230, the passive valve layer 134-1, the base plate 232, and the passive valve layer 134-2 may be stacked on top of each other. At least a portion of the pump 220 may be inserted into the recess of a frame of a fluidic manifold, and a perimeter portion of the actuator 230 may be coupled (e.g., welded) to a portion of the fluidic manifold.

The actuator 230 may include a flexible member that bends or changes shape based on the application or removal of a voltage. The actuator 230 may be one or more multiple components that are coupled together. In some examples, the actuator 230 includes one or more disc-shaped elements. In some examples, the actuator 230 includes an activation element (e.g., a piezo element with one or more electrodes) and an actuator diaphragm coupled to the activation element. A voltage applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump 220.

The base plate 232 may be a disc-shaped metal element. The base plate 232 may include a first hole 240 (e.g., a first through-hole) and a second hole 242 (e.g., a second through-hole). The first hole 240 (or the second hole 242) may be an inlet port, and the second hole 242 (or the first hole 240) may be an outlet port. The base plate 232 includes a first surface 231 and a second surface 233 disposed opposed to the first surface. The distance between the first surface 231 and the second surface 233 may define a thickness of the base plate 232.

The pump 220 includes a passive valve layer 234-1 and a passive valve layer 234-2. In some examples, the passive valve layer 234-1 is a circular disc member (e.g., a metal-based material). The passive valve layer 234-1 includes a fluid control element 238 at a location on the passive valve layer 134-1. The fluid control element 238 may be a feature on the passive valve layer 234-1 that causes fluid to flow through the passive valve layer 234-1 in one direction (but not the other direction). In some examples, the fluid control element 238 is a check valve. In some examples, the fluid control element 238 includes an input check valve. In some examples, the fluid control element 238 includes an output check valve. The fluid control element 238 may include one or more slots. In some examples, the fluid control element 238 includes a helical slot. In some examples, the fluid control element 238 is aligned with the second hole 242. The passive valve layer 234-1 includes a hole 236 (e.g., a through-hole). In some examples, the hole 236 has a size (e.g., a diameter) that is less than the size (e.g., the diameter) of the first hole 240 or the second hole 242. In some examples, the hole 236 is aligned with the first hole 240.

In some examples, the passive valve layer 234-2 is a circular disc member. The passive valve layer 234-2 includes a fluid control element 244 at a location on the passive valve layer 234-2. The fluid control element 244 may be a feature on the passive valve layer 234-2 that causes fluid to flow through the passive valve layer 234-2 in one direction (but not in the other direction). In some examples, the fluid control element 244 is a check valve. In some examples, the fluid control element 244 includes an input check valve. In some examples, the fluid control element 244 includes an output check valve. The fluid control element 244 may include one or more slots. In some examples, the fluid control element 244 includes a helical slot. In some examples, the fluid control element 244 is aligned with the first hole 240. The passive valve layer 234-2 includes a hole 246 (e.g., a through-hole). In some examples, the hole 246 has a size (e.g., a diameter) that is less than the size (e.g., the diameter) of the first hole 240 or the second hole 242. In some examples, the hole 246 is aligned with the second hole 242.

The passive valve layer 234-1 may contact the first surface 231 of the base plate 232. The passive valve layer 234-2 may contact the second surface 233 of the base plate 232. The location of the passive valve layer 234-2 (e.g., below the second surface 233) may increase the stroke of the pump 220. For example, a longer stroke (e.g., distance that the actuator diaphragm can travel) may allow the pump 220 to draw in and expel a larger volume of fluid with each cycle.

In some examples, as shown in FIG. 2B, two passive valve layers are located below the base plate 232. For example, the actuator 230 may contact (e.g., directly contact) the base plate 232 (e.g., the first surface 231 of the base plate 232), and the passive valve layer 234-2 may contact (e.g., directly contact) the base plate 232 (e.g., the second surface 233 of the base plate 232). The passive valve layer 234-1 may contact (e.g., directly contact) the surface (e.g., the bottom surface) of the passive valve layer 234-2.

In some examples, as shown in FIG. 2C, a passive valve layer (e.g., the passive valve layer 234-1 or the passive valve layer 234-2) may define a double valve, e.g., a valve portion 235 disposed on a valve seat 269 defining an opening 237. The valve portion 235 may be a smaller circular portion defining a helical valve that is inserted into and disposed on top of the valve seat 269. The passive valve layer with the double valve may be the passive valve layer 234-1 and/or the passive valve layer 234-2 of FIGS. 2A and/or 2B.

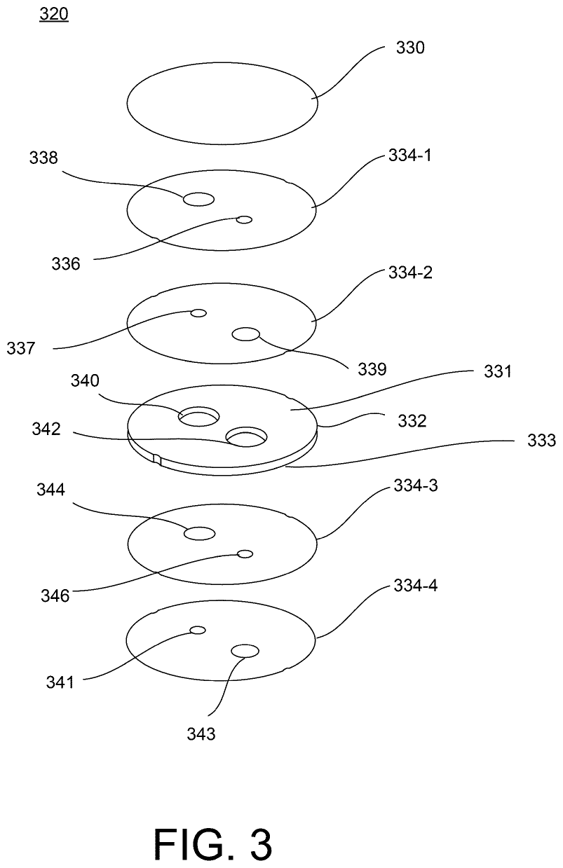

FIG. 3 illustrates a pump 320 according to an aspect. The pump 320 may be an example of the pump 120 of FIG. 1 and/or the pump 220 of FIGS. 2A to 2C and may include any of the details discussed herein. The pump 320 may include an actuator 330, a passive valve layer 334-1, a passive valve layer 334-2, a base plate 332, a passive valve layer 334-3, and a passive valve layer 334-4. The actuator 330, the passive valve layer 334-1, the passive valve layer 334-2, the base plate 332, the passive valve layer 334-3, and the passive valve layer 334-4 may be stacked on top of each other. At least a portion of the pump 320 may be inserted into the recess (e.g., a circular opening) of a frame of a fluidic manifold, and a perimeter portion of the actuator 330 may be coupled (e.g., welded) to a portion of the fluidic manifold.

The actuator 330 may include a flexible member that bends or changes shape based on the application or removal of a voltage. The actuator 330 may be one or more multiple components that are coupled together. In some examples, the actuator 330 includes one or more disc-shaped elements. In some examples, the actuator 330 includes an activation element (e.g., a piezo element with one or more electrodes) and an actuator diaphragm coupled to the activation element. A voltage applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump 320.

The base plate 332 may be a disc-shaped metal element. The base plate 332 may include a first hole 340 (e.g., a first through-hole) and a second hole 342 (e.g., a second through-hole). The first hole 340 (or the second hole 342) may be an inlet port, and the second hole 342 (or the first hole 340) may be an outlet port. The base plate 332 includes a first surface 331 and a second surface 333 disposed opposed to the first surface. The distance between the first surface 331 and the second surface 333 may define a thickness of the base plate 332.

The pump 320 includes two passive valve layers on one side of the base plate 332 and two passive valve layers on the other side of the base plate 332. For example, the pump 320 includes a passive valve layer 334-1 and a passive valve layer 334-2. The passive valve layer 334-1 and the passive valve layer 334-2 may be disposed between the base plate 332 and the actuator 330. The passive valve layer 334-2 may contact a first surface 331 of the base plate 332 (e.g., sit on top of the base plate 332), and the passive valve layer 334-1 may contact the passive valve layer 334-2 (e.g., sit on top of the passive valve layer 334-2).

In some examples, the passive valve layer 334-1 is a circular disc member. The passive valve layer 334-1 includes a fluid control element 338 at a location on the passive valve layer 334-1. The fluid control element 338 may be a feature on the passive valve layer 334-1 that causes fluid to flow through the passive valve layer 334-1 in one direction (but not the other direction). In some examples, the fluid control element 338 is a check valve. In some examples, the fluid control element 338 includes an input check valve. In some examples, the fluid control element 338 includes an output check valve. The fluid control element 338 may include one or more slots. In some examples, the fluid control element 338 includes a helical slot. In some examples, the fluid control element 338 is aligned with the first hole 340 of the base plate 332. The passive valve layer 334-1 includes a hole 336 (e.g., a through-hole). In some examples, the hole 336 has a size (e.g., a diameter) that is less than the size (e.g., the diameter) of the first hole 340 or the second hole 342 of the base plate 332. In some examples, the hole 336 is aligned with the second hole 342.

In some examples, the passive valve layer 334-2 is a circular disc member. The passive valve layer 334-2 includes a fluid control element 339 at a location on the passive valve layer 334-2. The fluid control element 339 may be a feature on the passive valve layer 334-2 that causes fluid to flow through the passive valve layer 334-2 in one direction (but not the other direction). In some examples, the fluid control element 339 is a check valve. In some examples, the fluid control element 339 includes an input check valve. In some examples, the fluid control element 339 includes an output check valve. The fluid control element 339 may include one or more slots. In some examples, the fluid control element 339 includes a helical slot. In some examples, the fluid control element 339 is aligned with the second hole 342 of the base plate 332. The passive valve layer 334-2 includes a hole 337 (e.g., a through-hole). In some examples, the hole 337 has a size (e.g., a diameter) that is less than the size (e.g., the diameter) of the first hole 340 or the second hole 342 of the base plate 332. In some examples, the hole 337 is aligned with the first hole 340 of the base plate 332.

The pump 320 includes a passive valve layer 334-3 and a passive valve layer 334-4. The passive valve layer 334-3 and the passive valve layer 334-4 may be disposed on the other side (e.g., the second surface 333) of the base plate 332. The passive valve layer 334-3 may contact the second surface 333 of the base plate 332 (e.g., the base plate 332 may sit on top of the passive valve layer 334-3), and the passive valve layer 334-4 may contact the passive valve layer 334-3 (e.g., the passive valve layer 334-3 may sit on top of the passive valve layer 334-4).

In some examples, the passive valve layer 334-3 is a circular disc member. The passive valve layer 334-3 includes a fluid control element 344 at a location on the passive valve layer 334-3. The fluid control element 344 may be a feature on the passive valve layer 334-3 that causes fluid to flow through the passive valve layer 334-3 in one direction (but not in the other direction). In some examples, the fluid control element 344 is a check valve. In some examples, the fluid control element 344 includes an input check valve. In some examples, the fluid control element 344 includes an output check valve. The fluid control element 344 may include one or more slots. In some examples, the fluid control element 344 includes a helical slot. In some examples, the fluid control element 344 is aligned with the first hole 340. The passive valve layer 334-3 includes a hole 346 (e.g., a through-hole). In some examples, the hole 346 has a size (e.g., a diameter) that is less than the size (e.g., the diameter) of the first hole 340 or the second hole 342. In some examples, the hole 346 is aligned with the second hole 342.

In some examples, the passive valve layer 334-4 is a circular disc member. The passive valve layer 334-4 includes a fluid control element 343 at a location on the passive valve layer 334-4. The fluid control element 343 may be a feature on the passive valve layer 334-4 that causes fluid to flow through the passive valve layer 334-4 in one direction (but not the other direction). In some examples, the fluid control element 343 is a check valve. In some examples, the fluid control element 343 includes an input check valve. In some examples, the fluid control element 343 includes an output check valve. The fluid control element 343 may include one or more slots. In some examples, the fluid control element 343 includes a helical slot. In some examples, the fluid control element 343 is aligned with the second hole 342 of the base plate 332. The passive valve layer 334-4 includes a hole 341 (e.g., a through-hole). In some examples, the hole 341 has a size (e.g., a diameter) that is less than the size (e.g., the diameter) of the first hole 340 or the second hole 342 of the base plate 332. In some examples, the hole 341 is aligned with the first hole 340 of the base plate 332.

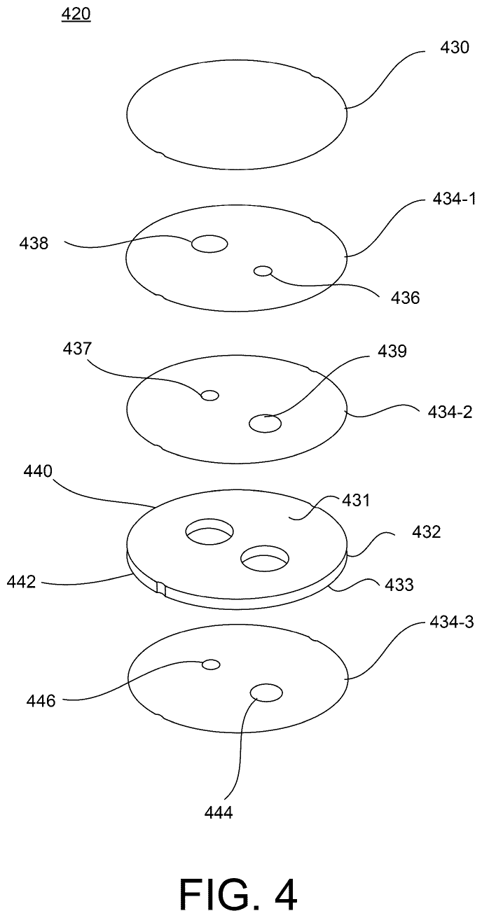

FIG. 4 illustrates a pump 420 according to an aspect. The pump 420 may be an example of the pump 120 of FIG. 1, the pump 220 of FIGS. 2A to 2C, and/or the pump 320 of FIG. 3 and may include any of the details discussed herein. The pump 420 may include two passive valve layers on one side of the base plate 432, and one passive valve layer on the other side of the base plate 432.

The pump 420 includes an actuator 430. The actuator 430 may include a flexible member that bends or changes shape based on the application or removal of a voltage. The actuator 430 may be one or more multiple components that are coupled together. In some examples, the actuator 430 includes one or more disc-shaped elements. In some examples, the actuator 430 includes an activation element (e.g., a piezo element with one or more electrodes) and an actuator diaphragm coupled to the activation element. A voltage applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump 420.

The pump 420 includes a base plate 432. The base plate 432 may be a disc-shaped metal element. The base plate 432 includes a first hole 440 (e.g., a first through-hole) and a second hole 442 (e.g., a second through-hole). The first hole 440 (or the second hole 442) may be an inlet port, and the second hole 442 (or the first hole 440) may be an outlet port. The base plate 432 includes a first surface 431 and a second surface 433 disposed opposed to the first surface. The distance between the first surface 431 and the second surface 433 may define a thickness of the base plate 432.

The pump 420 includes two passive valve layers on one side of the base plate 432 and one passive valve layer on the other side of the base plate 432. For example, the pump 420 includes a passive valve layer 434-1 and a passive valve layer 434-2. The passive valve layer 434-1 and the passive valve layer 434-2 are disposed between the base plate 432 and the actuator 430. The passive valve layer 434-2 may contact a first surface 431 of the base plate 432 (e.g., sit on top of the base plate 432), and the passive valve layer 434-1 may contact the passive valve layer 434-2 (e.g., sit on top of the passive valve layer 434-2). The passive valve layer 434-1 may contact the actuator 430.

In some examples, the passive valve layer 434-1 is a circular disc member. The passive valve layer 434-1 includes a fluid control element 438 at a location on the passive valve layer 434-1. The fluid control element 438 may be a feature on the passive valve layer 434-1 that causes fluid to flow through the passive valve layer 434-1 in one direction (but not the other direction). In some examples, the fluid control element 438 is a check valve. In some examples, the fluid control element 438 includes an input check valve. In some examples, the fluid control element 438 includes an output check valve. The fluid control element 438 may include one or more slots. In some examples, the fluid control element 438 includes a helical slot. In some examples, the fluid control element 438 is aligned with the first hole 440 of the base plate 432. The passive valve layer 434-1 includes a hole 436 (e.g., a through-hole). In some examples, the hole 436 has a size (e.g., a diameter) that is less than the size (e.g., the diameter) of the first hole 440 or the second hole 442 of the base plate 432. In some examples, the hole 436 is aligned with the second hole 442.

In some examples, the passive valve layer 434-2 is a circular disc member. The passive valve layer 434-2 includes a fluid control element 439 at a location on the passive valve layer 434-2. The fluid control element 439 may be a feature on the passive valve layer 434-2 that causes fluid to flow through the passive valve layer 434-2 in one direction (but not the other direction). In some examples, the fluid control element 439 is a check valve. In some examples, the fluid control element 439 includes an input check valve. In some examples, the fluid control element 439 includes an output check valve. The fluid control element 439 may include one or more slots. In some examples, the fluid control element 439 includes a helical slot. In some examples, the fluid control element 439 is aligned with the second hole 442 of the base plate 432. The passive valve layer 434-2 includes a hole 437 (e.g., a through-hole). In some examples, the hole 437 has a size (e.g., a diameter) that is less than the size (e.g., the diameter) of the first hole 440 or the second hole 442 of the base plate 432. In some examples, the hole 437 is aligned with the first hole 440 of the base plate 432.

The pump 420 includes a passive valve layer 434-3. The passive valve layer 434-3 may be disposed on the other side (e.g., the second surface 433) of the base plate 432. The passive valve layer 434-3 may contact (e.g., directly contact) the second surface 433 of the base plate 432 (e.g., the base plate 432 may sit on top of the passive valve layer 434-3).

In some examples, the passive valve layer 434-3 is a circular disc member. The passive valve layer 434-3 includes a fluid control element 444 at a location on the passive valve layer 434-3. The fluid control element 444 may be a feature on the passive valve layer 434-3 that causes fluid to flow through the passive valve layer 434-3 in one direction (but not in the other direction). In some examples, the fluid control element 444 is a check valve. In some examples, the fluid control element 444 includes an input check valve. In some examples, the fluid control element 444 includes an output check valve. The fluid control element 444 may include one or more slots. In some examples, the fluid control element 444 includes a helical slot. In some examples, the fluid control element 444 is aligned with the first hole 440. The passive valve layer 434-3 includes a hole 446 (e.g., a through-hole). In some examples, the hole 446 has a size (e.g., a diameter) that is less than the size (e.g., the diameter) of the first hole 440 or the second hole 442. In some examples, the hole 446 is aligned with the second hole 442.

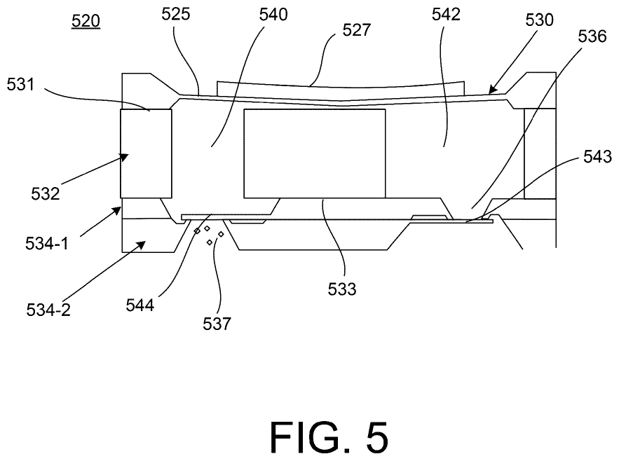

FIG. 5 illustrates a pump 520 for an electronic pump device of an inflatable medical device. The pump 520 may be an example of the pump 120 of FIG. 1, the pump 220 of FIGS. 2A to 2C, the pump 320 of FIG. 3, and/or the pump 420 of FIG. 4, and may include any of the details discussed with reference to those figures.

The pump 520 includes an actuator 530. The actuator 530 may include a flexible member that bends or changes shape based on the application or removal of a voltage. The actuator 330 includes an activation element 527 (e.g., a piezo element with one or more electrodes) and an actuator diaphragm 525 coupled to the activation element 527. A voltage applied to the activation element 527 may cause the activation element 527 to deform, thereby deforming the actuator diaphragm 525 (e.g., bend, change shape). For example, when an electrical signal is applied to the electrodes of the activation element 527, the activation element 527 undergoes a physical deformation, and this deformation is transmitted to the actuator diaphragm 525, causing it to move. The movement of the actuator diaphragm 525 controls the flow of fluid through the pump 520. In some examples, the actuator 530 is coupled to a base plate 532. In some examples, one or more passive valve layers may be disposed between the actuator 530 and the base plate 532.

The base plate 532 may include a first hole 540 (e.g., a first through-hole) and a second hole 542 (e.g., a second through-hole). The first hole 540 (or the second hole 542) may be an inlet port, and the second hole 542 (or the first hole 540) may be an outlet port. The base plate 532 includes a first surface 531 and a second surface 533 disposed opposed to the first surface. The distance between the first surface 531 and the second surface 533 may define a thickness of the base plate 532.

The pump 520 may include two passive valve layers on the backside (e.g., the second surface 533) of the base plate 532. For example, the pump 520 includes a passive valve layer 534-1 and a passive valve layer 534-2. The passive valve layer 534-1 may contact (e.g., directly contact) the second surface 533 of the base plate 532. The passive valve layer 534-2 may contact (e.g., directly contact) the passive valve layer 534-1.

In some examples, the passive valve layer 534-1 is a circular disc member. The passive valve layer 534-1 includes a fluid control element 544 at a location on the passive valve layer 534-1. The fluid control element 544 may be a feature on the passive valve layer 534-1 that causes fluid to flow through the passive valve layer 534-1 in one direction (e.g., upward direction). In some examples, the fluid control element 544 is a check valve. In some examples, the fluid control element 544 includes an input check valve. In some examples, the fluid control element 544 includes an output check valve. The fluid control element 544 may include one or more slots. In some examples, the fluid control element 544 includes a helical slot. In some examples, the fluid control element 544 is aligned with the first hole 540 of the base plate 532. The passive valve layer 534-1 includes a hole 536 (e.g., a through-hole). In some examples, the hole 536 has a size (e.g., a diameter) that is less than the size (e.g., the diameter) of the first hole 540 or the second hole 542 of the base plate 532. In some examples, the hole 536 is aligned with the second hole 542 of the base plate 532.

In some examples, the passive valve layer 534-2 is a circular disc member. The passive valve layer 534-2 includes a fluid control element 543 at a location on the passive valve layer 534-2. The fluid control element 543 may be a feature on the passive valve layer 534-2 that causes fluid to flow through the passive valve layer 534-2 in one direction (e.g., the downward direction-from the perspective of the figure). In some examples, the fluid control element 543 is a check valve. In some examples, the fluid control element 543 includes an input check valve. In some examples, the fluid control element 543 includes an output check valve. The fluid control element 543 may include one or more slots. In some examples, the fluid control element 543 includes a helical slot. In some examples, the fluid control element 543 is aligned with the second hole 542 of the base plate 532. The passive valve layer 534-2 includes a hole 537 (e.g., a through-hole). In some examples, the hole 537 has a size (e.g., a diameter) that is less than the size (e.g., the diameter) of the first hole 540 or the second hole 542 of the base plate 532. In some examples, the hole 537 is aligned with the first hole 540 of the base plate 532.

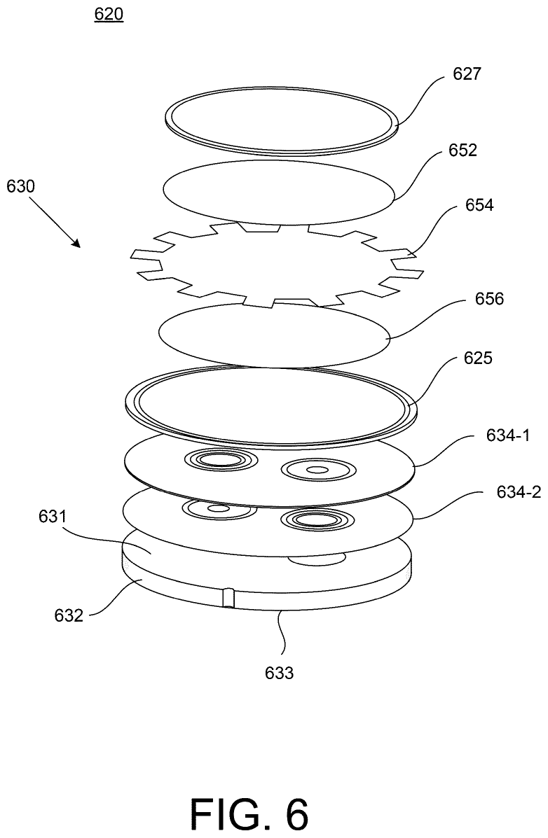

FIG. 6 illustrates an example of a pump 620 according to another aspect. The pump 620 may be an example of the pump 120 of FIG. 1, the pump 220 of FIGS. 2A to 2C, the pump 320 of FIG. 3, the pump 420 of FIG. 4, and/or the pump 520 of FIG. 5, and may include any of the details discussed with reference to those figures. Although FIG. 6 illustrates two passive valve layers (e.g., 634-1, 634-2) between a base plate 632 and an actuator 630, the pump 620 may include one or more passive valve layers on a second surface 633 of the base plate 632 as shown in FIGS. 1 to 5.

The pump 620 includes an actuator 630. The actuator 630 includes an activation element 627 (e.g., a piezo element), an adhesive layer 652, an isolation layer 654, an adhesive layer 656, and an actuator diaphragm 625. In some examples, the actuator 630 does not include an isolation layer 654. In some examples, the adhesive layer 652 includes an epoxy material. In some examples, the adhesive layer 656 includes an epoxy material.

The pump 620 includes a passive valve layer 634-1, a passive valve layer 634-2, and a base plate 632. In examples, the passive valve layer 634-1 is configured as an inlet valve, and the passive valve layer 634-2 is configured as an outlet valve. The base plate 632 includes a first surface 631 and a second surface 633. In some examples, the passive valve layer 634-2 contacts (e.g., directly contacts) the first surface 631 (or a portion thereof) of the base plate 632. In some examples, the passive valve layer 634-1 contacts (e.g., directly contacts) the passive valve layer 634-2. In some examples, the passive valve layer 634-1 and the passive valve layer 634-2 are located between the actuator diaphragm 625 and the base plate 632.

In some examples, the passive valve layer 634-1 contacts (e.g., directly contacts) the first surface 631 (or a portion thereof) of the base plate 632. In some examples, the passive valve layer 634-1 contacts (e.g., directly contacts) the actuator diaphragm 625. In some examples, the passive valve layer 634-2 contacts (e.g., directly contacts) the second surface 633 (or a portion thereof) of the base plate 632. In some examples, one or more passive valve layers are located on one side of the base plate 632 and one or more passive valve layers are located on the other side of the base plate 632.

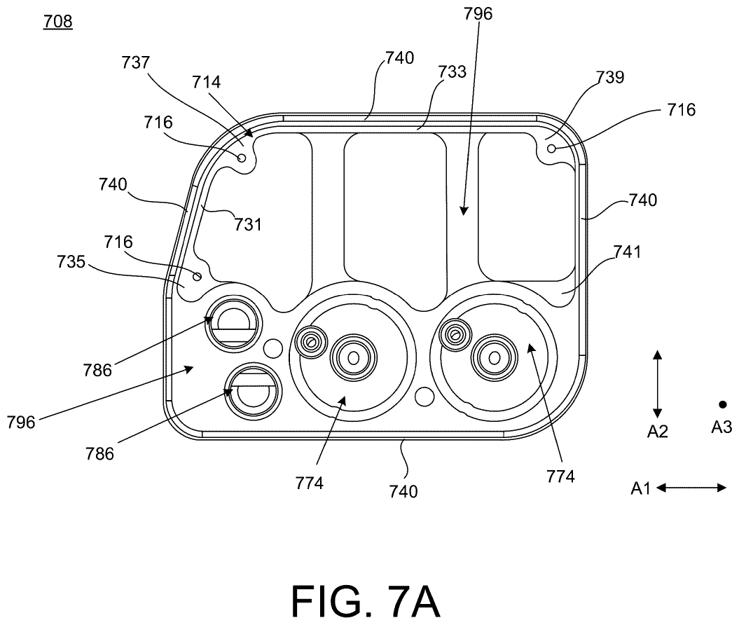

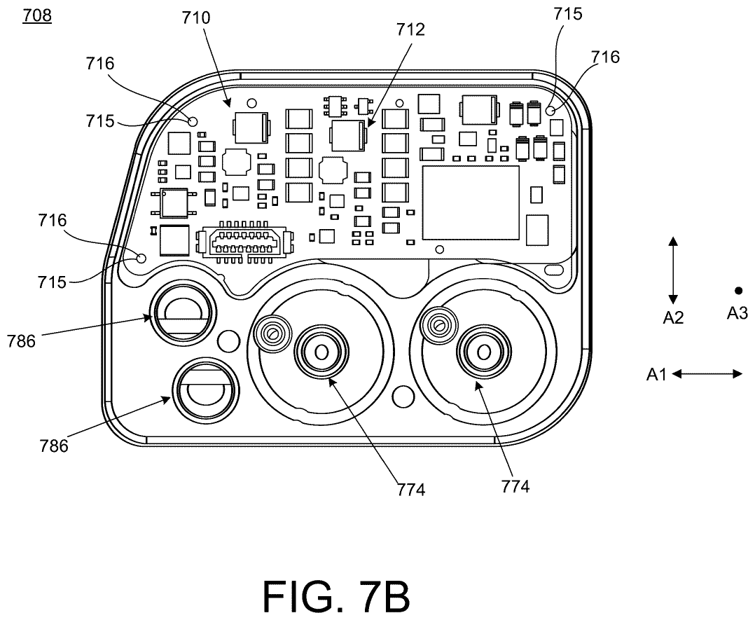

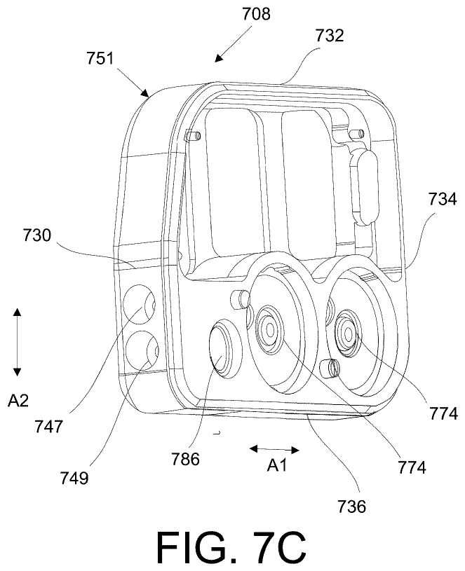

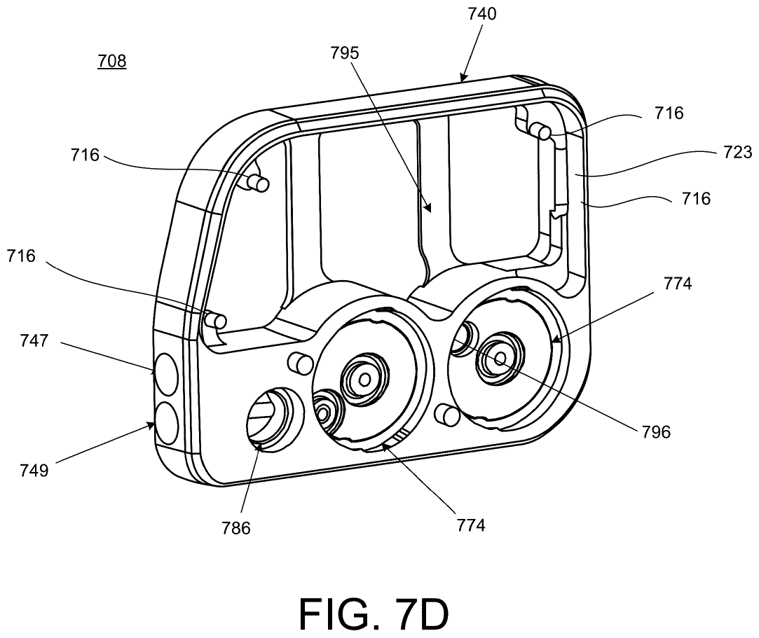

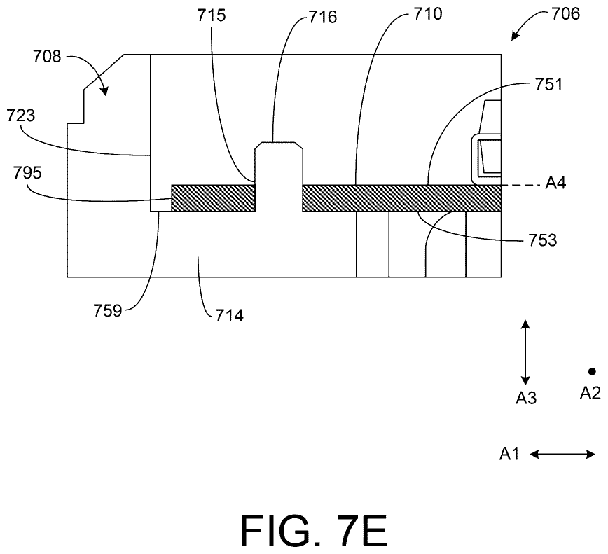

FIGS. 7A to 7E illustrates a fluidic manifold 708 of an electronic pump device 706 according to an aspect. The fluidic manifold 708 is configured to attach one or more fluidic components 774 such as the pump 120 of FIG. 1, the pump 220 of FIGS. 2A to 2C, the pump 320 of FIG. 3, the pump 420 of FIG. 4, the pump 520 of FIG. 5, and/or the pump 620 of FIG. 6.

In some examples, the fluidic manifold 708 includes a conductive-based material (e.g., a metal-based material). In some examples, the fluidic manifold 708 is a titanium frame. The fluidic manifold 708 may attach a circuit substrate 710 with electronic components. The fluidic manifold 708 may attach one or more fluidic components 774. A fluidic component 774 may be a pump. A fluidic component 774 may be a valve. The pump may be any of the pumps described in this disclosure. The fluidic manifold 708 includes a portion 796 configured to attach one or more pressure sensors 786 and one or more fluidic components 774 (e.g., pump(s), valves). The fluidic manifold 708 includes a portion 796 configured to attach a circuit substrate 710 with electronic components 712. In some examples, the circuit substrate 710 is a printed circuit board (PCB).

In some examples, the pressure sensors 786 include a first pressure sensor connected to a fluid reservoir (e.g., the fluid reservoir 102 of FIG. 1) to detect a pressure in the fluid reservoir, and a second pressure sensor connected to an inflatable member (e.g., the inflatable member 104 of FIG. 1) to detect a pressure in the inflatable member.

The fluidic manifold 108 includes a frame 740 with an inside edge 723. The fluidic manifold 708 includes a shelf 714 that extends from the inside edge 723. The circuit substrate 710 contacts the shelf 714 (e.g., a surface 759 of the shelf 714). The circuit substrate 710 may sit on top of the shelf 714 such that the circuit substrate 710 is positioned within the frame 740. The circuit substrate 710 includes a first surface 751 and a second surface 753 that is opposite to the first surface 751. As shown in FIG. 7E, the first surface 751 of the circuit substrate 710 is disposed in the plane A4. The thickness of the circuit substrate 710 is defined as the distance between the first surface 751 and the second surface 753 in the direction A3. The circuit substrate 710 includes an edge 795. The direction A3 is orthogonal to the plane A4. The direction A1 is perpendicular to the direction A3 and perpendicular to the direction A2. The direction A2 is perpendicular to the direction A1 and perpendicular to the direction A3. The second surface 753 of the circuit substrate 710 may contact at least a portion of the surface 759 of the shelf 714.

The fluidic manifold 708 includes one or more coupling members 716 that couples the circuit substrate 710 to the fluidic manifold 708 (e.g., to the shelf 714 of the fluidic manifold 708). In some examples, the coupling members 716 includes one or more male features (e.g., protrusions, posts, fasteners, rivets, etc.) and one or more female features (e.g., holes, slots, openings, etc.). The male feature may be defined on the fluidic manifold 708 (e.g., the shelf 714 of the fluidic manifold 708) and the female features may be circuit substrate 710. In some examples, the female feature may be defined on the fluidic manifold 708 (e.g., the shelf 714 of the fluidic manifold 708) and the male features may be defined on the circuit substrate 710.

In some examples, the coupling members 716 include protrusions (e.g., posts, cylindrical posts, grooved posts, extension members, etc.) that extend from the shelf 714 in the direction A3, and the protrusions extend through holes 715 on the circuit substrate 710. In some examples, the interaction between the protrusions and the holes 715 form a press-fit coupling mechanism. Although some examples use a press-fit coupling mechanism, the circuit substrate 710, and the fluidic manifold 708 may be coupled to each other based on other types of coupling mechanisms.

The shelf 714 may include one or more shelf portions (e.g., also referred to as shoulder portions) that extend from the inside edge 723 of the frame 740 in the directions A1 and A2. The shelf 714 may have a thickness that extends in the direction A3. In some examples, the shelf portions extend from the inside edge 723 in the direction A2 and/or the direction A3 at multiple different lengths. The shelf portions may include a corner portion 735, a corner portion 737, a corner portion 739, and a corner portion 741. The corner portion 735, the corner portion 737, and the corner portion 741 may define, include, or contact a coupling member 716. The shelf portions may include a connecting portion 731 that extends between the corner portion 735 and the corner portion 737. The shelf portions may include a connecting portion 733 that extends between the corner portion 737 and the corner portion 739.

The frame 740 may define a peripheral wall formed by a wall portion 730, a wall portion 732, a wall portion 734, and a wall portion 736. In some examples, the outer surface of the wall portion 730, the wall portion 732, the wall portion 734, and the wall portion 736 form a portion of the outer surface of the electronic pump device. The fluidic manifold 708 includes a first fluid port 747 and a second fluid port 749. The first fluid port 747 and the second fluid port 749 are defined on the wall portion 730. A first tube member (e.g., tube member 103 of FIG. 1) may be coupled to and extend from the first fluid port 747, and a second tube member (e.g., tube member 105 of FIG. 1) may be coupled to and extend from the second fluid port 749. In some examples, the outer surface of the wall portion 730, the wall portion 732, the wall portion 734, and the wall portion 736 form a portion of the outer surface of the housing (e.g., the housing 119 of FIG. 1).

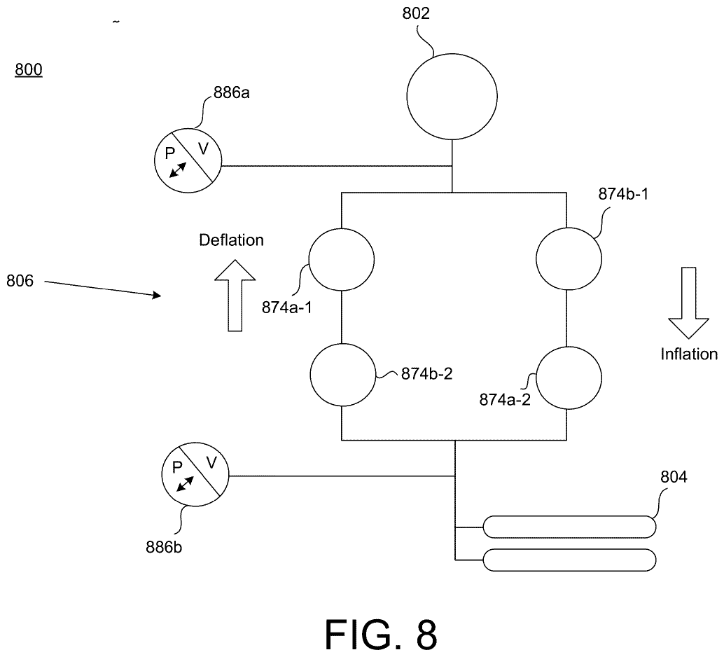

FIG. 8 illustrates an inflatable medical device 800 according to an aspect. In some examples, the implantable medical device 800 is an inflatable penile prosthesis. The implantable medical device 800 includes a fluid reservoir 802, an inflatable member 804, and an electronic pump device 806 configured to transfer fluid between the fluid reservoir 802 and the inflatable member 804 to inflate or deflate the inflatable member 804. In some examples, the inflatable member 804 is a penile inflation member (e.g., one or more inflatable cylinders) that may be implanted into the corpus cavernosum of the user.

The electronic pump device 806 includes a pressure sensor 886a connected to the fluid reservoir 802 and configured to monitor a pressure of the fluid reservoir 802, and a pressure sensor 886b connected to the inflatable member 804 and configured to monitor a pressure of the inflatable member 804. The electronic pump device 806 includes a valve 874a-1 and a pump 874b-2. The pump 874b-2 may be any of the pumps described with reference to FIGS. 1 to 6. The valve 874a-1 and the pump 874b-2 may be used to deflate the inflatable member 804. The electronic pump device 806 includes a valve 874a-2 and a pump 874b-1. The valve 874a-2 and the pump 874b-1 may be used to inflate the inflatable member 804. The pump 874b-1 may be any of the pumps described with reference to FIGS. 1 to 6.

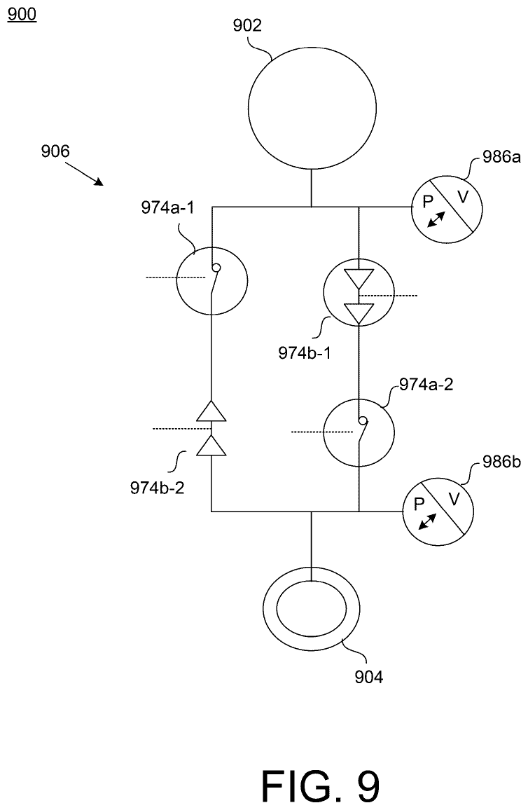

FIG. 9 illustrates an inflatable medical device 900 according to an aspect. In some examples, the implantable medical device 900 is an artificial urinary sphincter device. The implantable medical device 900 includes a fluid reservoir 902, an inflatable member 904, and an electronic pump device 906 configured to transfer fluid between the fluid reservoir 902 and the inflatable member 904 to inflate or deflate the inflatable member 904. In some examples, the inflatable member 904 is an inflatable cuff.

The electronic pump device 906 includes a pressure sensor 986a connected to the fluid reservoir 902 and configured to monitor a pressure of the fluid reservoir 902, and a pressure sensor 986b connected to the inflatable member 904 and configured to monitor a pressure of the inflatable member 904. The electronic pump device 906 includes a valve 974a-1 and a pump 974b-2. The valve 974a-1 and the pump 974b-2 may be used to deflate the inflatable member 904. The pump 974b-2 may be any of the pumps described with reference to FIGS. 1 to 6. The electronic pump device 906 includes a valve 974b-1 and a pump 974b-1. The valve 974a-2 and the pump 974b-1 may be used to inflate the inflatable member 904. The pump 974b-1 may be any of the pumps described with reference to FIGS. 1 to 6.

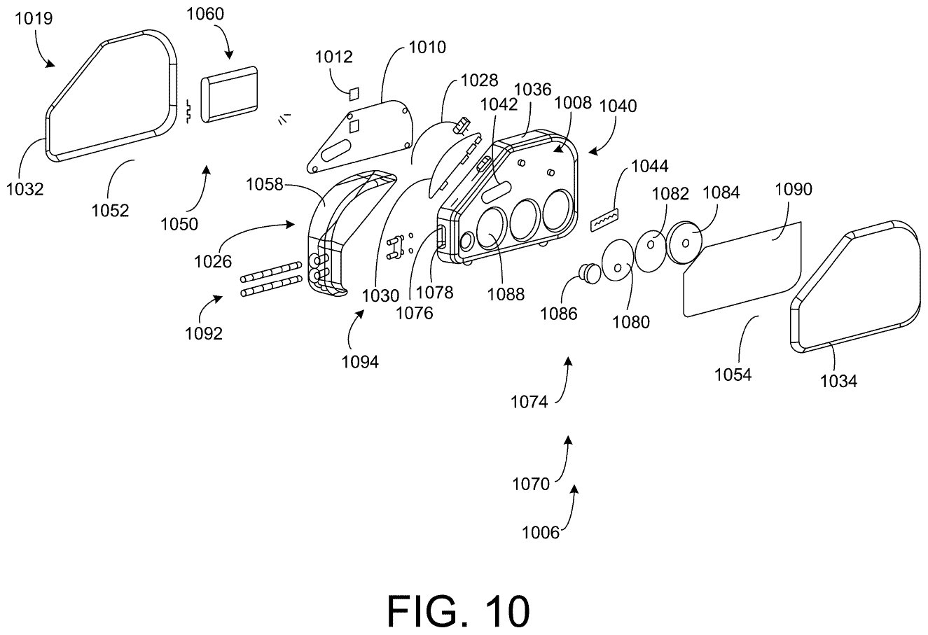

FIG. 10 illustrates an example of an exploded view of an electronic pump device 1006 according to an aspect. The housing 1019 includes a first sidewall 1032, a second sidewall 1034, a peripheral wall 1036, and a frame 1040. The first sidewall 1032, second sidewall 1034, and the peripheral wall 1036 are hermetically sealed together to form an internal compartment 1050 within the housing 1019.

The frame 1040 is disposed within the internal compartment 1050 to form a first partition 1052 and a second partition 1054 in such a manner that the first partition 1052 is hermetically sealed from the second partition 1054. The frame 1040 can be integrally formed with the peripheral wall 1036, the first sidewall 1032, and/or the second sidewall 1034. In some examples, the frame 1040 is welded to the peripheral wall 1036 or welded to the first sidewall 1032, and/or the second sidewall 1034. The first sidewall 1032, the peripheral wall 1036, and the frame 1040 may form the first partition 1052. The second sidewall 1034, the peripheral wall 1036, and the frame 1040 may form the second partition 1054, which is opposite the frame 1040 from the first partition 1052.

The electronic pump device 1006 can include a header 1026 attached to the housing 1019 to form an internal region 1058 between an inner surface of the header 1026 and an outer surface of the housing 1019 that includes power and communication interface structures such as a secondary coil 1028 and the antenna 1030 external to the hermetically sealed housing 1019. The header 1026 is configured from a dielectric or insulative material, such as a radome, to allow the transmission of power and communication signals between the antenna 1030 and a handset programmer or charger, and between the secondary coil 1028 and the charger. For example, the header 1026 may include an over-molded polymer affixed to the housing 1019 and including the secondary coil 1028 and the antenna 1030 within the internal region 1058. The secondary coil 1028 and antenna 1030 are constructed from a biocompatible material. In some examples, the secondary coil 1028 and antenna 1030 can be formed as a coil from a stamped titanium core clad with gold or silver. In some examples, the secondary coil 1028 and antenna 1030 can be formed from a gold wire.

The electronic pump device 1006 includes an energy storage system, such as a battery (e.g., a rechargeable power source) (e.g., a rechargeable battery), and electronic components 1012 within the first partition 1052. The electronic components 1012 can be disposed on a circuit substrate 1010, such as a plurality of circuit boards, within the first partition 1052. The battery 1060 can assume various forms appropriate to provide power for generating desired electrical signals and to store power provided from the electronic components 1012. For example, the battery 1060 can incorporate lithium-ion (Li+) chemistry, e.g., a lithium-ion battery to operate the electronic components 1012. In some examples, the electronic components 1012 can be implemented by various components including resistors, capacitors, transistors, and integrated circuits disposed on the circuit substrate 1010. The secondary coil 1028 and antenna 1030 are electrically coupled to the electronic components 1012 within the first partition 1052, such as via a hermetic feedthrough component.

The electronic components 1012 can include a recharge system, a communication system, and a controller. The recharge system includes hardware configured to interface with the secondary coil 1028 to receive power signals, and to provide the power signals in a form suitable to recharge the battery 1060 and can include circuitry to reduce the likelihood of overcharging the battery 1060. The communication system includes hardware configured to interface with the antenna 1030 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., external device 101) (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna 1030 for transmission. In some examples, the communication system can be configured to receive and transmit radio frequency signals via the antenna 1030. The controller can include a microcontroller to operate the recharge system and to receive and operate in response to communication signals or generate communication signals from the communication system.

The electronic pump device 1006 also includes a fluidic circuit 1070 within the second partition 1054 and opposite the frame 1040 from the battery 1060 and electronic components 1012. In some examples, the frame 1040 can include an opening 1042 that includes a hermetic interface 1044, such as a feedthrough hermetically affixed to the frame 1040. The electronic components 1012 are operably coupled to the fluidic circuit 1070 across the frame 1040 via the hermetic interface 1044. For example, the controller of the electronic components 1012, powered by the battery 1060, can cause the operation of the fluidic circuit 1070 such as to control and monitor the fluidic circuit 1070.

The fluidic circuit 1070 includes a fluidic manifold 1008 and fluidic components 1074 operably coupled to the fluidic manifold 1008. In some examples, the fluidic manifold 1008 is a structure integrated into the frame 1040 such that the fluidic manifold 1008 and the frame 1040 together form the hermetic barrier between the first partition 1052 and the second partition 1054 of the internal compartment 1050. For instance, the battery 1060, the circuit substrate 1010, or electronic components 1012 can be coupled to a first major surface of the fluidic manifold 1008 in the first partition 1052, and the fluidic components 1074 are operably coupled to a second, and opposite major surface of the fluidic manifold 1008 in the second partition 1054.

The fluidic circuit 1070 provides for the transfer of the fluid between the fluid reservoir (e.g., the fluid reservoir 102 of FIG. 1) and the inflatable member (e.g., the inflatable member 104 of FIG. 1). The fluidic manifold 1008, which can be a hermetic manifold, segments and contains the fluid from the internal compartment 1050 to reduce the chance of fluid exchange and directs the fluid from a first port 1076 to a second port 1078 via internal fluid passageways or channels.

The fluidic components 1074 include a plurality of fluid pumps, such as pumps 1080, 1082, a valve 1084 mounted into the fluidic manifold 1008 in fluidic communication with a manifold passageway to transfer fluid from the first port 1076 to the second port 1078. The pump 1080 or the pump 1082 may be any of the pumps described with reference to FIGS. 1 to 6. The pumps and the valve(s) are in fluid communication with a single fluid passageway between ports 1076, 1078. The fluidic components 1074 also includes one or more pressure sensors 1086 operably coupled to the fluidic manifold 1008 and in fluidic communication with the passageway to detect a pressure of the fluid within the fluidic manifold 1008.

The fluidic components 1074 are included in a planar configuration on the fluidic manifold 1008 in which the pumps 1080, 1082, valve 1084, and pressure sensor 1086 are mounted into the fluidic manifold 1008 on a plane for slim profile within the second partition 1054. The fluidic manifold 1008 can include chambers 1088 formed into the second major surface in which the chambers are fluidically coupled to the single passageway within the fluidic manifold 1008. The chambers are configured to receive the pumps 1080, 1082, and valve 1084 and one or more pressure sensors 1086. In some examples, the fluidic manifold 1008 can receive a piezoelectric pump. The fluidic manifold 1008 can receive a component cover 1090 over the fluidic components 1074, which can be hermetically sealed to the second major surface.

In some examples, the electronic pump device 1006 may include kink resistant tubing 1092 that can extend through the header 1026 and attached to the ports 1076, 1078 via components such as a barb 1094 and O-rings. The kink resistant tubing 1092 can be attached to the tube members 103, 105 of FIG. 1 to fluidically couple the electronic pump device 1006 to the fluid reservoir (e.g., the fluid reservoir 102 of FIG. 1) and the inflatable member (e.g., the inflatable member 104 of FIG. 1).

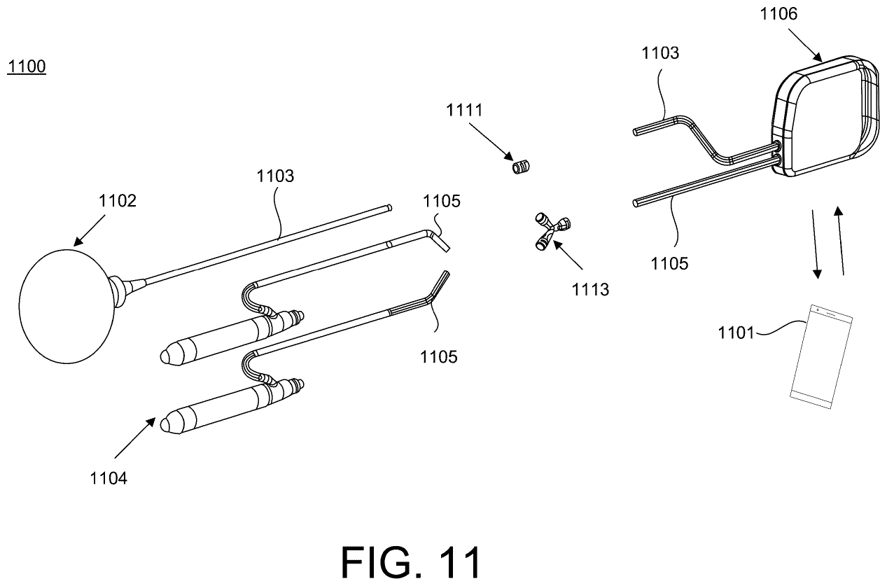

FIG. 11 illustrates a perspective of an inflatable penile prosthesis 1100 according to an aspect. The inflatable penile prosthesis 1100 may be an example of any of the medical devices discussed herein (e.g., including implantable medical device 100), and, therefore, may include any of the details discussed with reference to the previous figures.

The inflatable penile prosthesis 1100 includes an inflatable member 1104, a fluid reservoir 1102, and an electronic pump device 1106. The inflatable member 1104 includes a pair of inflatable cylinders. The electronic pump device 1106 may be an example of any of the pump devices discussed with reference to the previous figures and may include any of the details discussed herein. The electronic pump device 1106 includes fluidic components such as pumps, valves, and/or sensing devices positioned in fluid passageways. The electronic pump device 1106 includes components such as, for example, one or more fluid control devices, one or more pressure sensors, and other such components. The electronic pump device 1106 includes an electronic control system configured to provide for the transfer of fluid between a fluid reservoir 1102 and an inflatable member 1104 via the fluidic components.

The electronic pump device 1106 may include one or more integrated circuits. In some examples, the integrated circuits are included in a printed circuit board that is included in a housing of the electronic pump device 1106. Fluidic components and the electronic components of the electronic pump device 1106 are included in a housing. In some examples, fluidic components and electronic components in the housing define a manifold (e.g., an electronically controlled fluidic manifold) that provides for the electronic control of the flow of fluid between the fluid reservoir 1102 and the inflatable member 1104. In some examples, the electronic pump device 1106 can communicate with an external device 1101, via respective communication modules. For example, an application stored in a memory and executed by a processor of the external device 1101 may allow the user and/or a physician to operate, view, monitor and alter operation of the inflatable penile prosthesis 1100.

The inflatable penile prosthesis 1100 includes one or more first tube members 1103 that connect a first fluid port of the electronic pump device 1106 with the fluid reservoir 1102. One or more second tube members 1105 connect a second fluid port of the electronic pump device 1106 with the inflatable member 1104 in the form of the inflatable cylinders. In some examples, the inflatable penile prosthesis 1100 includes a connector 1111 that is used to connect two tube members 1103 together, and a connector 1113 that is used to connect two tube members 1105 together.

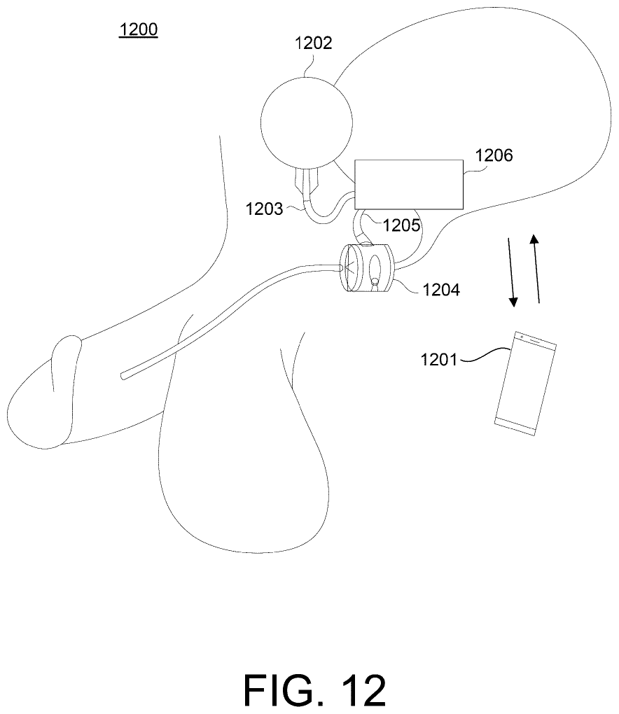

FIG. 12 illustrates a urinary control device 1200 having an electronic pump device 1206 according to an aspect. The urinary control device 1200 may be an example of the implantable medical device 100. In some examples, the urinary control device 1200 is an artificial urinary sphincter device. The electronic pump device 1206 may include any of the features of the pump devices discussed herein. The urinary control device 1200 includes an electronic pump device 1206, a fluid reservoir 1202, and a cuff 1204 (e.g., an inflatable cuff).

The fluid reservoir 1202 may be a pressure-regulating inflation balloon or element. The fluid reservoir 1202 is in operative fluid communication with the cuff 1204 via one or more tube members 1203, 1205. The fluid reservoir 1202 is constructed of polymer material that is capable of elastic deformation to reduce fluid volume within the fluid reservoir 1202 and push fluid out of the fluid reservoir 1202 and into the cuff 1204. However, the material of the fluid reservoir 1202 can be biased or include a shape memory construct adapted to generally maintain the fluid reservoir 1202 in its expanded state with a relatively constant fluid volume and pressure. In some examples, this constant level of pressure exerted from the fluid reservoir 1202 to the cuff 1204 will keep the cuff 1204 at a desired inflated state when open fluid communication is provided between the fluid reservoir 1202 and the cuff 1204. In some examples, the fluid reservoir 1202 is implanted into the abdominal space.

A user may use an external device 1201 to control the urinary control device 1200. In some examples, the user may use the external device 1201 to inflate or deflate the cuff 1204. For example, in response to the user activating an inflation cycle using the external device 1201, the external device 1201 may transmit a wireless signal to the electronic pump device 1206 to initiate the inflation cycle to transfer fluid from the fluid reservoir 1202 to the cuff 1204 (e.g., by opening an active valve where the pressure in the fluid reservoir 1202 causes the fluid to move through the active valve to the cuff 1204). In some examples, in response to the user activating a deflation cycle using the external device 1201, the external device 1201 may transmit a wireless signal to the pump device 1206 to initiate the deflation cycle to transfer fluid from the cuff 1204 to the fluid reservoir 1202.

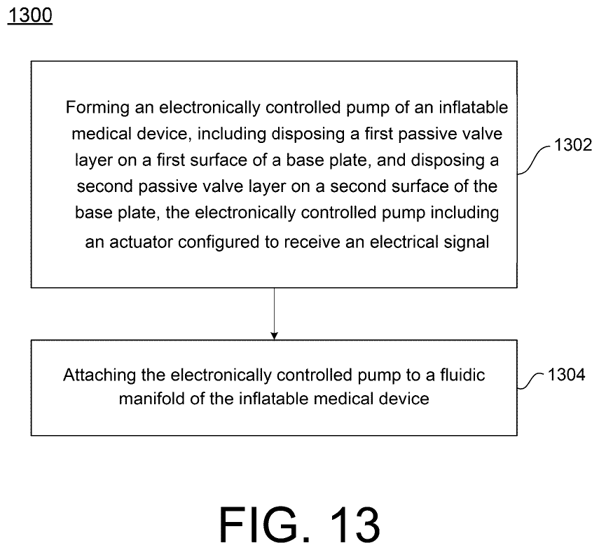

FIG. 13 illustrates a flowchart 1300 depicting example operations of coupling a fluidic component to a fluidic manifold according to an aspect. Although the flowchart 1300 of FIG. 13 illustrates the operations in sequential order, it will be appreciated that this is merely an example, and that additional or alternative operations may be included. Further, operations of FIG. 13 and related operations may be executed in a different order than that shown, or in a parallel or overlapping fashion.

Operation 1302 includes forming an electronically controlled pump of an inflatable medical device, including disposing a first passive valve layer on a first surface of a base plate and disposing a second passive valve layer on a second surface of the base plate, the electronically controlled pump including an actuator configured to receive an electrical signal. Operation 1304 includes attaching the electronically controlled pump to a fluidic manifold of the inflatable medical device.

Clause 1. An implantable medical device comprising: an inflatable member; a fluid reservoir; and an electronic pump device configured to transfer fluid between the inflatable member and the fluid reservoir, the electronic pump device including: an actuator; a base plate having a first surface and a second surface, the first surface of the base plate facing the actuator; a passive valve layer contacting the second surface of the base plate.

Clause 2. The implantable medical device of clause 1, wherein the passive valve layer is a first passive valve layer, the electronic pump device including: a second passive valve layer contacting the first surface of the base plate or the first passive valve layer.

Clause 3. The implantable medical device of clause 2, wherein the first passive valve layer includes a first flow control element, and the second passive valve layer includes a second flow control element.

Clause 4. The implantable medical device of clause 3, wherein the base plate includes a first hole and a second hole, the first flow control element being aligned with the first hole, the second flow control element being aligned with the second hole.

Clause 5. The implantable medical device of any one of clauses 1 to 4, wherein the actuator is coupled to the passive valve layer.

Clause 6. The implantable medical device of clause 1, wherein the passive valve layer is a first passive valve layer, the electronic pump device including: a second passive valve layer contacting the first surface of the base plate; a third passive valve layer contacting the second passive valve layer, the actuator contacting the third passive valve layer.

Clause 7. The implantable medical device of clause 6, wherein the electronic pump device includes: a fourth passive valve layer contacting the first passive valve layer.

Clause 8. The implantable medical device of any one of clauses 1 to 7, wherein the actuator includes an activation element and an actuator diaphragm coupled to the activation element, the activation element configured to receive an electrical signal, wherein, in response to the electrical signal, the activation element is configured to deform.

Clause 9. The implantable medical device of any one of clauses 1 to 8, wherein the passive valve layer defines a helical slot.

Clause 10. An electronic pump device for an inflatable medical device, the electronic pump device including: an actuator; a base plate having a first surface and a second surface, the first surface of the base plate facing the actuator; a passive valve layer contacting the second surface of the base plate.

Clause 11. The electronic pump device of clause 10, wherein the passive valve layer is a first passive valve layer, the electronic pump device including: a second passive valve layer contacting the first surface of the base plate or the first passive valve layer.

Clause 12. The electronic pump device of clause 11, wherein the first passive valve layer includes a first flow control element, and the second passive valve layer includes a second flow control element, wherein the base plate includes a first hole and a second hole, the first flow control element being aligned with the first hole, the second flow control element being aligned with the second hole.

Clause 13. A method comprising: forming an electronically controlled pump of an inflatable medical device, including: disposing a first passive valve layer on a first surface of a base plate; and disposing a second passive valve layer on a second surface of the base plate, the electronically controlled pump including an actuator configured to receive an electrical signal; and attaching the electronically controlled pump to a fluidic manifold of the inflatable medical device.

Clause 14. The method of clause 13, wherein the first passive valve layer includes a first flow control element, and the second passive valve layer includes a second flow control element, wherein the base plate includes a first hole and a second hole, the method further comprising: aligning the first flow control element with the first hole; and aligning the second flow control element with the second hole.

Clause 15. The method of clause 13 or 14, further comprising: disposing a third passive valve layer on the first passive valve layer, the actuator contacting the third passive valve layer; and disposing a fourth passive valve layer on the second passive valve layer.

Clause 16. An implantable medical device comprising: an inflatable member; a fluid reservoir; and an electronic pump device configured to transfer fluid between the inflatable member and the fluid reservoir, the electronic pump device including: an actuator; a base plate having a first surface and a second surface, the first surface of the base plate facing the actuator; a passive valve layer contacting the second surface of the base plate.

Clause 17. The implantable medical device of clause 16, wherein the passive valve layer is a first passive valve layer, the electronic pump device including: a second passive valve layer contacting the first surface of the base plate or the first passive valve layer.

Clause 18. The implantable medical device of clause 17, wherein the first passive valve layer includes a first flow control element, and the second passive valve layer includes a second flow control element.

Clause 19. The implantable medical device of clause 18, wherein the base plate includes a first hole and a second hole, the first flow control element being aligned with the first hole, the second flow control element being aligned with the second hole.

Clause 20. The implantable medical device of clause 16, wherein the actuator is coupled to the passive valve layer.

Clause 21. The implantable medical device of clause 16, wherein the passive valve layer is a first passive valve layer, the electronic pump device including: a second passive valve layer contacting the first surface of the base plate; a third passive valve layer contacting the second passive valve layer, the actuator contacting the third passive valve layer.

Clause 22. The implantable medical device of clause 21, wherein the electronic pump device includes: a fourth passive valve layer contacting the first passive valve layer.

Clause 23. The implantable medical device of clause 16, wherein the actuator includes an activation element and an actuator diaphragm coupled to the activation element, the activation element configured to receive an electrical signal, wherein, in response to the electrical signal, the activation element is configured to deform.

Clause 24. The implantable medical device of clause 16, wherein the passive valve layer defines a helical slot.

Clause 25. An electronic pump device for an inflatable medical device, the electronic pump device including: an actuator having an activation element and an actuator diaphragm coupled to the activation element; a base plate having a first surface and a second surface; a first passive valve layer contacting the first surface of the base plate, the first passive valve layer being located between the base plate and the actuator diaphragm; and a second passive valve layer contacting the second surface of the base plate.

Clause 26. The electronic pump device of clause 25, wherein the first passive valve layer includes a first flow control element, and the second passive valve layer includes a second flow control element.

Clause 27. The electronic pump device of clause 26, wherein the first flow control element includes a first helical slot at a first location on the first passive valve layer, and the second flow control element includes a second helical slot at a second location on the second passive valve layer.

Clause 28. The electronic pump device of clause 26, wherein the base plate includes a first hole and a second hole, the first hole extending through the first surface of the base plate and the second surface of the base plate, the first flow control element being aligned with the first hole of the base plate, the second flow control element being aligned with the second hole of the base plate.

Clause 29. The electronic pump device of clause 28, wherein the first passive valve layer includes a third hole, and the second passive valve layer includes a fourth hole, the third hole being aligned with the second hole of the base plate, the fourth hole being aligned with the first hole of the base plate.

Clause 30. The electronic pump device of clause 25, wherein the electronic pump device includes: a third passive valve layer contacting the first passive valve layer, the actuator diaphragm contacting the third passive valve layer.

Clause 31. The electronic pump device of clause 30, wherein the electronic pump device includes: a fourth passive valve layer contacting the second passive valve layer.

Clause 32. The electronic pump device of clause 25, wherein the first passive valve layer defines an inlet valve, and the second passive valve layer defines an outlet valve.

Clause 33. A method comprising: forming an electronically controlled pump of an inflatable medical device, including: disposing a first passive valve layer on a first surface of a base plate; and disposing a second passive valve layer on a second surface of the base plate, the electronically controlled pump including an actuator configured to receive an electrical signal; and attaching the electronically controlled pump to a fluidic manifold of the inflatable medical device.

Clause 34. The method of clause 33, wherein the first passive valve layer includes a first flow control element, and the second passive valve layer includes a second flow control element, wherein the base plate includes a first hole and a second hole, the method further comprising: aligning the first flow control element with the first hole; and aligning the second flow control element with the second hole.

Clause 35. The method of clause 33, further comprising: disposing a third passive valve layer on the first passive valve layer, the actuator contacting the third passive valve layer; and disposing a fourth passive valve layer on the second passive valve layer.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir; and
an electronic pump device configured to transfer fluid between the inflatable member and the fluid reservoir, the electronic pump device including:
an actuator;
a base plate having a first surface and a second surface, the first surface of the base plate facing the actuator;
a passive valve layer contacting the second surface of the base plate.

2. The implantable medical device of claim 1, wherein the passive valve layer is a first passive valve layer, the electronic pump device including:
a second passive valve layer contacting the first surface of the base plate or the first passive valve layer.

3. The implantable medical device of claim 2, wherein the first passive valve layer includes a first flow control element, and the second passive valve layer includes a second flow control element.

4. The implantable medical device of claim 3, wherein the base plate includes a first hole and a second hole, the first flow control element being aligned with the first hole, the second flow control element being aligned with the second hole.

5. The implantable medical device of claim 1, wherein the actuator is coupled to the passive valve layer.

6. The implantable medical device of claim 1, wherein the passive valve layer is a first passive valve layer, the electronic pump device including:
a second passive valve layer contacting the first surface of the base plate;
a third passive valve layer contacting the second passive valve layer, the actuator contacting the third passive valve layer.

7. The implantable medical device of claim 6, wherein the electronic pump device includes:
a fourth passive valve layer contacting the first passive valve layer.

8. The implantable medical device of claim 1, wherein the actuator includes an activation element and an actuator diaphragm coupled to the activation element, the activation element configured to receive an electrical signal, wherein, in response to the electrical signal, the activation element is configured to deform.

9. The implantable medical device of claim 1, wherein the passive valve layer defines a helical slot.

10. An electronic pump device for an inflatable medical device, the electronic pump device including:
an actuator having an activation element and an actuator diaphragm coupled to the activation element;
a base plate having a first surface and a second surface;
a first passive valve layer contacting the first surface of the base plate, the first passive valve layer being located between the base plate and the actuator diaphragm; and
a second passive valve layer contacting the second surface of the base plate.

11. The electronic pump device of claim 10, wherein the first passive valve layer includes a first flow control element, and the second passive valve layer includes a second flow control element.

12. The electronic pump device of claim 11, wherein the first flow control element includes a first helical slot at a first location on the first passive valve layer, and the second flow control element includes a second helical slot at a second location on the second passive valve layer.

13. The electronic pump device of claim 11, wherein the base plate includes a first hole and a second hole, the first hole extending through the first surface of the base plate and the second surface of the base plate, the first flow control element being aligned with the first hole of the base plate, the second flow control element being aligned with the second hole of the base plate.

14. The electronic pump device of claim 13, wherein the first passive valve layer includes a third hole, and the second passive valve layer includes a fourth hole, the third hole being aligned with the second hole of the base plate, the fourth hole being aligned with the first hole of the base plate.

15. The electronic pump device of claim 10, wherein the electronic pump device includes:
a third passive valve layer contacting the first passive valve layer, the actuator diaphragm contacting the third passive valve layer.

16. The electronic pump device of claim 15, wherein the electronic pump device includes:
a fourth passive valve layer contacting the second passive valve layer.

17. The electronic pump device of claim 10, wherein the first passive valve layer defines an inlet valve, and the second passive valve layer defines an outlet valve.

18. A method comprising:
forming an electronically controlled pump of an inflatable medical device, including:
disposing a first passive valve layer on a first surface of a base plate; and
disposing a second passive valve layer on a second surface of the base plate, the electronically controlled pump including an actuator configured to receive an electrical signal; and
attaching the electronically controlled pump to a fluidic manifold of the inflatable medical device.

19. The method of claim 18, wherein the first passive valve layer includes a first flow control element, and the second passive valve layer includes a second flow control element, wherein the base plate includes a first hole and a second hole, the method further comprising:
aligning the first flow control element with the first hole; and
aligning the second flow control element with the second hole.

20. The method of claim 18, further comprising:
disposing a third passive valve layer on the first passive valve layer, the actuator contacting the third passive valve layer; and
disposing a fourth passive valve layer on the second passive valve layer.
